(19) United States
(12) Patent Application Publication
(10) Pub. No.: US 2022/0409419 A1
(43) Pub. Date: Dec. 29, 2022

(54) ODOR TREATMENT FOR A URINE COLLECTION SYSTEM

(71) Applicant: PureWick Corporation, El Cajon, CA (US)

(72) Inventors: Anthony Garvey, Listowel (IR); Meghan Caracciola, Washington, DC (US); Padraig Fogarty, Ballina-Killaloe (IR); Tamika Richardson, Schenectady, NY (US)

(21) Appl. No.: 17/809,083

(22) Filed: Jun. 27, 2022

Related U.S. Application Data
(60) Provisional application No. 63/215,607, filed on Jun. 28, 2021.

(51) Int. Cl. A61F 5/441; A61F 5/451; B01J 20/20; B01J 20/28; A61L 9/014

(52) U.S. Cl. CPC A61F 5/441; A61F 5/451; B01J 20/20; B01J 20/2808; B01J 20/28064; B01J 20/28066; A61L 9/014

(57) ABSTRACT

Embodiments are directed to a urine collection system for collecting and transporting urine away from the body. The system includes a urine collection device configured to be positioned at least proximate to a urethra of a user. The system includes a first tube in fluid communication with the urine collection device. The system includes a urine collection container configured to receive urine from the urine collection device. The urine collection container includes an exhaust port. The system also includes a second tube in fluid communication with the urine collection container and a pump configured to pull an at least partial vacuum in the urine collection container through the second tube to draw the urine from the urine collection device into the urine collection container. The pump includes an exhaust vent. The system also includes an odor filter located between the exhaust port and the exhaust vent.

### CROSS-REFERENCE TO RELATED APPLICATIONS

This application claims priority to U.S. Provisional Patent Application No. 63/215,607 filed on Jun. 28, 2021, the disclosure of which is incorporated herein, in its entirety, by this reference.

### BACKGROUND

A patient may have limited or impaired mobility such that typical urination processes are challenging or impossible. For example, the patient may have surgery or a disability that impairs mobility. In another example, the patient may have restricted travel conditions such as those experience by pilots, drivers, and workers in hazardous areas. Additionally, fluid collection from the patient may be needed for monitoring purposes or clinical testing.

Urine collection systems have been developed to address some of these circumstances. Bed pans and urinary catheters, such as a Foley catheter, may also be used. However, bed pans and urinary catheters have several problems associated therewith. For example, bed pans may be prone to discomfort, spills, and other hygiene issues. Urinary catheters be may be uncomfortable, painful, and may cause urinary tract infections. Other urine collection systems may restrict the mobility of the patent due to use of a stationary pump or other vacuum source to draw fluid into the collection system.

Urine also has an unpleasant odor due to odor-causing organic compounds that may be aliphatic, aromatic, or heterocyclic compounds containing oxygen, sulfur, or nitrogen. The odor-causing molecules can be masked using a more pleasant smelling molecule, such as a perfume.

Thus, users and manufacturers of fluid collection assemblies continue to seek new and improved devices, systems, and methods to collect urine.

### SUMMARY

Embodiments disclosed herein are related to urine collection systems and methods of using urine collection systems. In an embodiment, a urine collection system is disclosed. The urine collection system may include a urine collection device configured to be positioned at least proximate to a urethra of a user. The urine collection system may further include a first tube in fluid communication with the urine collection device and a urine collection container configured to receive urine from the urine collection device. The urine collection container may include an exhaust port. The urine collection system may further include a second tube in fluid communication with the urine collection container and a pump configured to pull an at least partial vacuum in the urine collection container through the second tube to draw the urine from the urine collection device into the urine collection container. In some embodiments, the pump may include an exhaust vent. The urine collection system may also include an odor filter located between the exhaust port and the exhaust vent.

In an embodiment, a method for minimizing urine odor of a urine collection system is disclosed. The method may include producing an at least partial vacuum to draw urine from a urine collection device through a tube into a urine collection container. The method also may include collecting urine in a substantially air-tight urine collection container and drawing air from the urine collection container through at least one odor filter.

Features from any of the disclosed embodiments may be used in combination with one another, without limitation. In addition, other features and advantages of the present disclosure will become apparent to those of ordinary skill in the art through consideration of the following detailed description and the accompanying drawings.

### BRIEF DESCRIPTION OF THE DRAWINGS

The drawings illustrate several embodiments of the present disclosure, wherein identical reference numerals refer to identical or similar elements or features in different views or embodiments shown in the drawings.

FIG. 1A is a block diagram of a urine collection system, according to an embodiment.

FIG. 1B is an isomeric view of a urine collection system, according to an embodiment.

FIG. 2A is a top view of a urine collection container, showing an exhaust port and odor filter on the container, according to an embodiment.

FIG. 2B is a side view of a portion of the urine collection container, showing an odor filter and housing for the odor filter, according to an embodiment.

FIG. 2C is an isometric view of a portion of the urine collection system, showing a pump exhaust vent and an odor filter, according to an embodiment.

FIG. 3A is an isometric view of an odor filter, according to an embodiment.

FIG. 3B is an isometric view of an odor filter, according to an embodiment.

FIG. 4 is a process flow diagram of a method for minimizing urine odor of a urine collection system, according to an embodiment

### DETAILED DESCRIPTION

Embodiments disclosed herein are related to devices, systems, and methods of using urine collection devices and systems. Water is the main component of urine, but urine also includes salt (sodium, potassium and chloride), uric acid and urea. Uric acid is a natural waste product from food digestion and urea is a waste product made of ammonia and carbon dioxide—all substances the body tries to get rid of through urine. In an embodiment, the urine collection system may include components or methods to minimize or eliminate the odor of urine when collected with the urine collection system. Users and caregivers, then, are benefited from a urine collection system that is configured to minimize the odor of the urine.

In some embodiments, the urine collection system includes a urine collection device configured to be positioned at least proximate to a urethra of a user and a tube in fluid communication with the urine collection device. The urine collection system may also include a urine collection container configured to receive fluid from the urine collection device and a pump configured to pull an at least partial vacuum within the urine collection system to draw fluid from the urine collection device through the tube into the urine collection container. The pump may draw suction from an exhaust port of the urine collection container and discharge the exhaust from an exhaust vent into the environment. The urine collection system may include an odor filter configured to capture and/or minimize odors from the urine collection system.

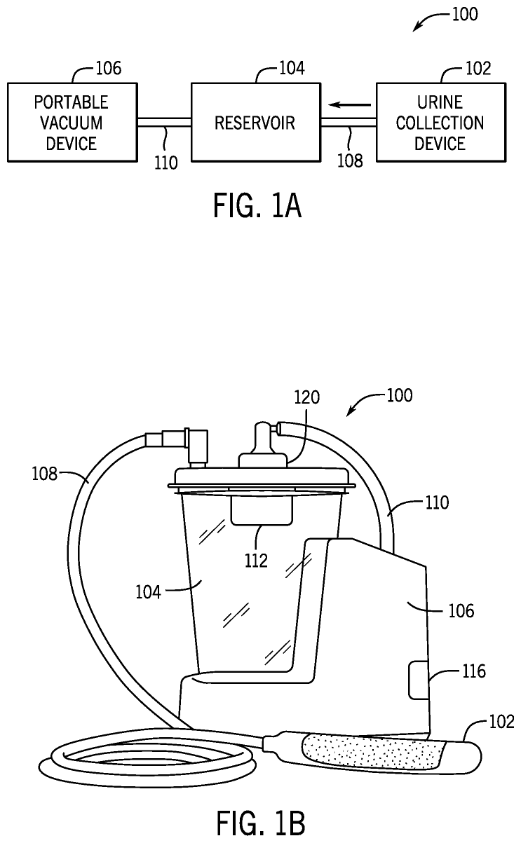

FIG. 1A is a block diagram of a urine collection system 100 and FIG. 1B is an isomeric view of the urine collection system 100, according to an embodiment. The urine collection system 100 may be included in embodiments of urine collection systems described herein. As shown in FIGS. 1A-1B, the system 100 includes a urine collection device 102 (e.g., any of the urine collection assemblies disclosed herein), a urine collection container 104 (or reservoir), and a portable vacuum device or pump 106. The urine collection device 102 and the urine collection container 104 may be fluidly coupled to each other via a first tube 108. The pump 106 and the urine collection container 104 may be fluidly coupled to each other via a second tube 110. In some embodiments, the pump 106 may be coupled directly to the urine collection container 104. The urine or other bodily fluids collected in the urine collection device 102 may be removed from the urine collection device 102 via the first tube 108 coupled to the urine collection device 102. Suction force may be introduced into the chamber of the urine collection device 102 via the inlet of the first tube 108 responsive to suction (e.g., vacuum) force applied at the outlet of the first tube 108.

The suction force may be applied to the outlet of the first tube 108 by the pump 106 either directly or indirectly. The suction force may be applied indirectly via the urine collection container 104. For example, the outlet of the first tube 108 may be disposed within or fluidly coupled to an interior region of the urine collection container 104 and the second tube 110 may extend from the urine collection container 104 to the pump 106. Accordingly, the pump 106 may apply suction to the urine collection device 102 via the urine collection container 14. In some embodiments, the suction force may be applied directly via the pump 106. For example, the outlet of the first tube 108 may be disposed within the pump 106. The second tube 110 may extend from the pump 106 to a point outside of the urine collection device 102, such as to the urine collection container 104. In such examples, the pump 106 may be disposed between the urine collection device 102 and the urine collection container 104.

The urine collection device 102 may be positioned at least proximate a urethra of a user. The urine collection device 102 may include a fluid impermeable enclosure and a fluid permeable membrane. The fluid impermeable enclosure at least partially defines a reservoir. The reservoir may define a volume for receiving and storing a liquid (e.g., urine, vaginal discharge, penile discharge, reproductive fluids, blood, sweat, wound discharge, interstitial fluid, cerebrospinal fluid, or other bodily fluids) and may be configured to receive fluid discharged from the user through the fluid permeable membrane. The fluid impermeable enclosure defines a perimeter and an exterior surface of the urine collection device 102. In some embodiments, the urine collection device 102 may also include a fluid permeable membrane. The permeable membrane may be configured to direct urine away from a portion of skin surrounding an anatomy of the user to provide a dry contact surface. The urine collection device 102 may be positioned in a manner to prevent or reduce leakage, promote an adjustable fit (e.g., variable contour to fit a user's of varying dimensions), or retain contact with the body when ambulatory.

The urine collection device 102 shown in FIG. 1B is an example of a female fluid collection device configured to collect fluid(s) from females (e.g., collect urine from a female urethra). Further examples of female fluid collection assemblies are disclosed in U.S. Pat. No. 10,390,989 issued on Aug. 27, 2019, the disclosure of which is incorporated herein, in its entirety, by this reference. However, the urine collection devices, systems, and methods disclosed herein may include male fluid collection assemblies and/or devices shaped, sized, and otherwise configured to collect fluid(s) from males (e.g., collect urine from a male urethra). Examples of male fluid collection assemblies are disclosed in U.S. Provisional Patent Applications No. 63/067,542 filed on Aug. 19, 2020 and U.S. patent application Ser. No. 16/433,773 filed on Jun. 6, 2019, the disclosure of which are incorporated herein, in its entirety, by this reference.

The pump 106 may be in fluid communication with an interior region of the urine collection container 104 and may be configured to pull at least a partial vacuum on the interior region of the urine collection container 104 effective to draw the urine from the urine collection device 102 through the first tube 108 into the urine collection container 104. The pump 106 may include one or more of a manual vacuum pump, and electric vacuum pump, a diaphragm pump, a centrifugal pump, a displacement pump, a magnetically driven pump, a peristaltic pump, or any pump configured to produce a vacuum. In some examples, the pump 106 may be powered by one or more of a power cord (e.g., connected to a power socket), one or more batteries, or even manual power (e.g., a hand operated vacuum pump). The pumps disclosed herein may include one or more of a switch, a button, a plug, a remote, or any other device suitable to activate the pump 106.

In some embodiments, the pump 106 may be coupled directly to the urine collection container 104. The urine collection container 104 may be sized and shaped to retain a fluid therein. The urine collection container 104 may include a bag (e.g., drainage bag), a bottle or cup (e.g., collection jar), or any other enclosed container for storing bodily fluid(s) such as urine. In some embodiments, the urine collection container 104 may include a generally rigid exterior housing and an interior portion configured to contain the fluid. The urine collection container 104 may be opaque or clear according to different embodiments and may include a generally rectangular front or rear profile or be cylindrical. The urine collection container 104 may be reusable and dishwasher safe, and may include a generally rigid material such as polycarbonate, plastic, rubber, metal, glass, combinations thereof, or any other suitable materials.

In some examples, the first tube 108 may extend from the urine collection device 102 and attach to the urine collection container 104 at a first point therein. The second tube 110 may extend from the pump 106 and attach to the urine collection container 104 at a second point therein. The second point may include an exhaust port 112. As shown in FIG. 1B, the exhaust port 112 may be on a lid 114 of the urine collection container 104. The exhaust port 112 may be configured to allow air to pass through the lid 114 and into the second tube 110 without allowing liquid to pass through. The second tube 110 may be coupled to the urine collection container 104 at the exhaust port 112 such that the pump 106 draws air from the urine collection container 104 through the exhaust port 112 and expels the air out of an exhaust vent 116. The first tube 108 and the second tube 110 may include a flexible tube. In some embodiments, at least a portion of the first tube 108 and/or second tube 110 may be substantially opaque, thereby inhibiting viewing of the urine within the tubes 108,110.

In some embodiments, the pump 106 may include the exhaust vent 116. The exhaust vent 116 vents the air drawn from the urine collection container 104 to the surrounding environment. In some embodiments, the exhaust vent 116 may be located on the pump 106. To prevent and/or minimize odors from the urine from escaping through the exhaust vent 116, an odor filter may be located between the exhaust port 112 and the exhaust vent 116. The odor filter may be configured to inhibit urine odor from exiting through the exhaust vent 116.

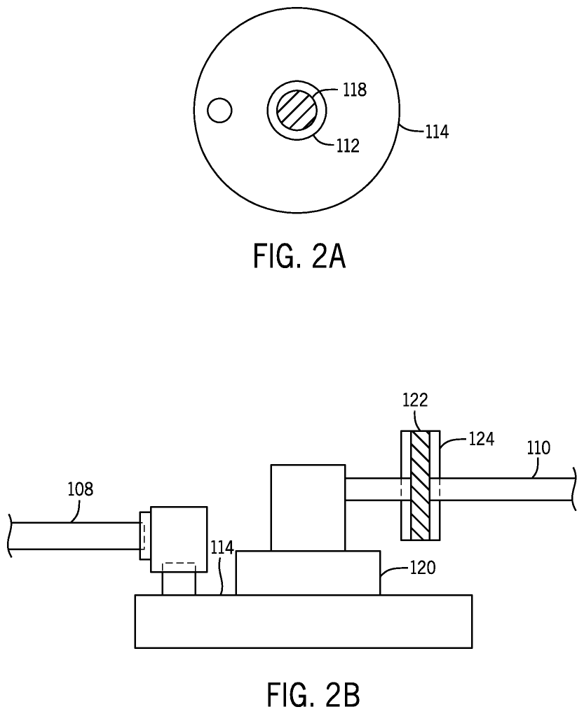

FIG. 2A is a top view of the urine collection container 104, showing the exhaust port 112 and an odor filter 118 on the lid 114 of the urine collection container 104, according to an embodiment. In some embodiments, the odor filter 118 may be located proximate to the exhaust port 112. The odor filter 118 may be removable and replaceable. To replace the odor filter 118, the second tube 110 may be disconnected from the urine collection container 104 to expose the odor filter 118. In some embodiments, the odor filter 118 may be disposed within a housing 120 (shown in FIG. 1B) to make the odor filter 118 easier to remove. In some embodiments, the housing 120 may be coupled to the second tube 110. The housing 120 may screw or twist off in some embodiments. The housing 120 may be coupled to the urine collection container 104 in an interference fit, in other embodiments. Generally, the housing 120 may be air tight, such that all of the air flows through the odor filter 118 to the exhaust vent 116.

FIG. 2B is a side view of a portion of the urine collection container 104, showing an odor filter 122 and housing 124 for the odor filter 122, according to an embodiment. The odor filter 122 may be an in-line filter disposed within the second tube 110. To ensure all of the odor-rich air passes through the odor filter 122, the odor filter 122 may be sized and dimensioned to have a cross-section greater than or about the same cross-section as the second tube 110. The odor filter 122 and housing 124 may be located any place along the length of the second tube 110. Generally, the odor filter 122 should be placed at a location that is easy to access. The odor filter 122 may be removable and replaceable.

FIG. 2C is an isometric view of a portion of the urine collection system 100, showing the exhaust vent 116 of the pump 106 and an odor filter 130, according to an embodiment. The odor filter may include a housing 132 to access the odor filter. In some embodiments, the housing 132 may include a door 134 or other retention mechanism that retains the odor filter 130 within the housing 132. The odor filter 130 may be positioned proximate to the exhaust vent 116. The odor filter 130 may be removable and replaceable.

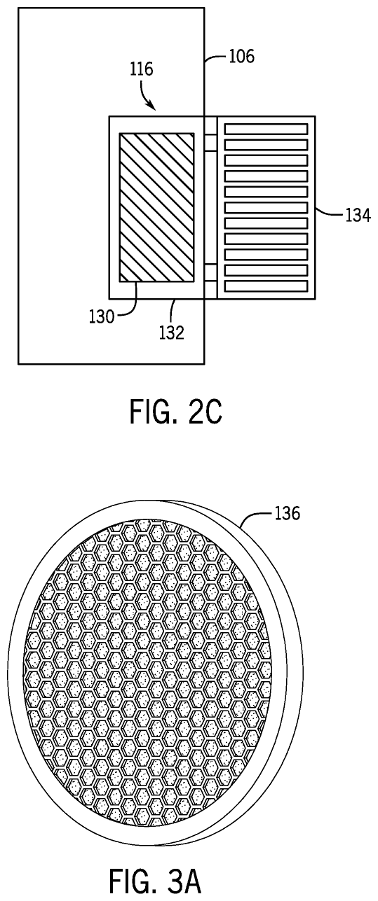

In some embodiments, the urine collection system 100 may include more than one odor filter. The odor filters may be in parallel or in series and located at locations that may be accessible. Referring now to FIGS. 3A-3B, in some embodiments, the odor filter 136 may be disk-shaped. In other embodiments, the odor filter 138 may be cylinder, rectangular, or cubic shaped. Some of the factors that may influence the shape of the odor filter used may include design airflow, filter design cost, filter replacement cost, filter efficiency, filter lifetime, and/or methods to replace the filter.

In some embodiments, the odor filter 136, 138 may include an activated carbon sorbent. In some embodiments, the activated carbon sorbent includes pores having a pore size of from about 5 Å to about 10 Å. In some embodiments, the pore size of the activated carbon sorbent may be from about 2 Å to about 15 Å. In some embodiments, the pore size of the activated carbon sorbent may be 2 Å or greater, such as about 5 Å or greater, about 6 Å or greater, about 7 Å or greater, about 8 Å or greater, about 9 Å or greater, about 10 Å or greater, about 13 Å or greater, or in ranges of about 2 Å to about 4 Å, about 4 Å to about 5 Å, about 5 Å to about 8 Å, about 8 Å to about 10 Å, about 10 Å to about 12 Å, or about 12 Å to about 15 Å. Adsorption pores are the only regions within an active carbon particle with sufficient adsorption forces to adsorb impurities (e.g. odors). They are the smallest pores within the particle, consisting of gaps between the graphite plates of about 1 to 5 molecular diameters in size. The finer the particle size of an activated carbon, the better the access to the surface area and the faster the rate of adsorption kinetics.

The activated carbon sorbent has a surface area of from about 500 m²/g to about 3,000 m²/g. In some embodiments, the pore volume of the activated carbon sorbent may be from about 500 m²/g to about 2,500 m²/g. In some embodiments, the pore volume may be about 500 m²/g or greater, such as about 600 m²/g or greater, about 900 m²/g or greater, about 1,200 m²/g or greater, about 2,000 m²/g or greater, about 2,300 m²/g or greater, about 2,500 m²/g or greater, about 2,800 m²/g or greater, or in ranges of about 500 m²/g to about 800 m²/g, about 800 m²/g to about 1,200 m²/g, about 1,200 m²/g to about 1800 m²/g, about 1,800 m²/g to about 2,400 m²/g, about 2,400 m²/g to about 2,800 m²/g, or about 2,800 m²/g to about 3,000 m²/g. Pore size distribution may describe the internal structures and adsorption capacities of an activated charcoal filter. Chemical functional groups and the internal surface areas account for, and are associated with, highly active surface properties of the activated charcoal. Due to varying methods of preparation, the pore sizes of the activated charcoal can be categorized as being micropores (width<2 nm), mesopores (width=2-50 nm), or macropores (width>50 nm); the differences in the size of their width openings being a representation of the pore distance.

The activated carbon sorbent contains pores having a pore volume of from about 0.2 cm³/g to about 1.0 cm³/g in the pore size range of from about 5 Å to about 10 Å. In some embodiments, the pore volume of the activated carbon sorbent may be from about 0.3 cm³/g to about 0.9 cm³/g. In some embodiments, the pore volume may be about 0.2 cm³/g or greater, such as about 0.4 cm³/g or greater, about 0.5 cm³/g or greater, about 0.6 cm³/g or greater, about 0.65 cm³/g or greater, about 0.7 cm³/g or greater, about 0.8 cm³/g or greater, about 0.9 cm³/g or greater, or in ranges of about 0.2 cm³/g to about 0.4 cm³/g, about 0.4 cm³/g to about 0.5 cm³/g, about 0.5 cm³/g to about 0.6 cm³/g, about 0.6 cm³/g to about 0.7 cm³/g, about 0.7 cm³/g to about 0.8 cm³/g, or about 0.8 cm³/g to about 1.0 cm³/g. The effectiveness at which activated carbon can remove contaminants from a stream is not based on the quantity of carbon, but, the activated carbon adsorption capacity. The greater the capacity, the more contaminants the activated carbon will be able to adsorb in volume.

In some embodiments, the odor filter 136, 138 may include a zeolite filter. Zeolite is a mineral that can form into a variety of structures made of arrays of aluminum, silica, and oxygen known as aluminosilicates. The most common is clinoptilolite. Their structure and composition make them microporous and inclined for cation exchange capabilities. The degree of such exchange properties vary from type to type and are noticeably weaker in naturally occurring zeolites due to potential impurities. However, zeolites can easily be produced synthetically with a heated solution of alumina, silica, and sodium hydroxide. Due to its high pore density, zeolite has a highly effective surface area. The zeolites may include pore sizes of 1-10 Å. Other filter materials may also be used.

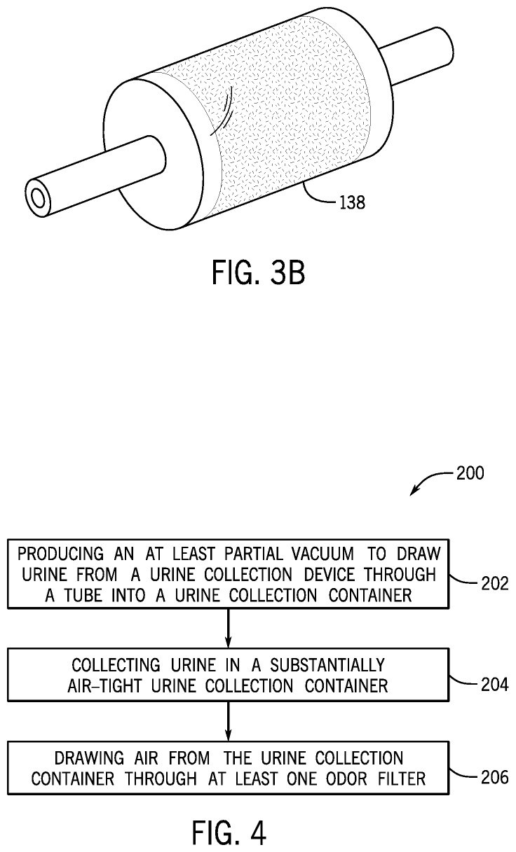

FIG. 4 is a process flow diagram of a method 200 for minimizing urine odor of a urine collection system, according to an embodiment. Some or all of the acts of method 200 may be performed using the urine collection system 100 and/or some of the components included in the urine collection system 100 discussed above. The method 200 may be initiated in response to a desire or need to remove the odor from the exhaust of a pump or vacuum device. At act 202, the method may include producing an at least partial vacuum to draw urine from a urine collection device through a tube into a urine collection container. The urine collection system may include a urine collection device configured to receive urine from a user. The method 200 may include an act 204, collecting urine in a substantially air-tight urine collection container. The urine collection container may be configured to store the urine collected from the urine collection device.

At act 206, air may be drawn from the urine collection container through at least one odor filter. In some embodiments, air may be drawn with a pump. In some embodiments, a vacuum may be drawn in the container, causing the urine to be drawn into the container. The odor filter may be located in either a filter housing or may be disposed in-line within the tube. The odor filter may be a disk-shaped inline filter located on an exhaust vent of the pump. In some embodiments, the odor filter may be removed and replaced periodically.

Acts 202, 204, and 206 of the method 200 are for illustrative purposes. For example, the acts may be performed in different orders, split into multiple acts, modified, supplemented, or combined. In an embodiment, one or more of the acts may be omitted from the method 200. While the specific embodiments discussed herein have been focused on the cleaning or configuration changes of a urine collection system, it will be understood that the methods, systems, and assemblies discussed herein could be applied to other devices.

While various aspects and embodiments have been disclosed herein, other aspects and embodiments are contemplated. The various aspects and embodiment disclosed herein are for purposes of illustration and are not intended to be limiting.

Terms of degree (e.g., “about,” “substantially,” “generally,” etc.) indicate structurally or functionally insignificant variations. In an example, when the term of degree is included with a term indicating quantity, the term of degree is interpreted to mean±10%, ±5%, or +2% of the term indicating quantity. In an example, when the term of degree is used to modify a shape, the term of degree indicates that the shape being modified by the term of degree has the appearance of the disclosed shape. For instance, the term of degree may be used to indicate that the shape may have rounded corners instead of sharp corners, curved edges instead of straight edges, one or more protrusions extending therefrom, is oblong, is the same as the disclosed shape, etc.

What is claimed is:

1. A urine collection system, comprising:
a urine collection device configured to be positioned at least proximate to a urethra of a user;
a first tube in fluid communication with the urine collection device;
a urine collection container configured to receive urine from the urine collection device, the urine collection container including an exhaust port;
a second tube in fluid communication with the urine collection container;
a pump configured to pull an at least partial vacuum in the urine collection container through the second tube to draw the urine from the urine collection device into the urine collection container, wherein the pump includes an exhaust vent; and
an odor filter located between the exhaust port and the exhaust vent.

2. The urine collection system of claim 1, wherein the odor filter is configured to inhibit urine odor from exiting through the exhaust vent.

3. The urine collection system of claim 1, wherein the second tube is coupled to the urine collection container at the exhaust port such that the pump draws air from the urine collection container through the exhaust port and expels the air out of the exhaust vent.

4. The urine collection system of claim 1, wherein the odor filter is located proximate to the exhaust port.

5. The urine collection system of claim 1, wherein the odor filter is an in-line filter disposed within the second tube.

6. The urine collection system of claim 5, wherein the odor filter is sized and dimensioned to have a cross-section about the same cross-section as the second tube.

7. The urine collection system of claim 1, wherein the odor filter is positioned proximate to the exhaust vent.

8. The urine collection system of claim 1, wherein the urine collection system further includes a filter housing configured to retain the odor filter.

9. The urine collection system of claim 1, wherein the odor filter is removable and replaceable.

10. The urine collection system of claim 1, wherein the odor filter is disk-shaped.

11. The urine collection system of claim 1, wherein the odor filter is cylindrical.

12. The urine collection system of claim 1, wherein the odor filter includes an activated carbon sorbent.

13. The urine collection system of claim 12, wherein the activated carbon sorbent includes pores having a pore size of from about 5 Å to about 10 Å.

14. The urine collection system of claim 12, wherein the activated carbon sorbent has a surface area of from about 500 m²/g to about 3,000 m²/g.

15. The urine collection system of claim 12, wherein the activated carbon sorbent contains pores having a pore volume of from about 0.2 cm³/g to about 1.0 cm³/g in the pore size range of from about 5 Å to about 10 Å.

16. The urine collection system of claim 1, wherein the odor filter includes a zeolite.

17. A method for minimizing urine odor of a urine collection system, the method comprising:
producing at least partial vacuum to draw the urine from a urine collection device through a tube into a urine collection container;
collecting urine in a substantially air-tight urine collection container; and
drawing air from the urine collection container through at least one odor filter.

18. The method of claim 17, wherein the odor filter is located in either a filter housing or disposed in-line within the tube.

19. The method of claim 17, further comprising removing and replacing the odor filter periodically.

20. The method of claim 17, wherein the at least partial vacuum is produced with a pump and the odor filter is a disk-shaped inline filter located on an exhaust vent of the pump.
